(19) United States
(12) Patent Application Publication
(10) Pub. No.: US 2022/0291230 A1
(43) Pub. Date: Sep. 15, 2022

(54) Method for Identifying Human Growth Hormone Proteoform (hGHP) Pattern Biomarker

(71) Applicant: Shandong First Medical University & Shandong Academy of Medical Sciences, Jinan City (CN)

(72) Inventors: Xianquan ZHAN, Jinan City (CN); Biao LI, Jinan City (CN); Xiaowei WANG, Jinan City (CN); Na LI, Jinan City (CN)

(73) Assignee: Shandong First Medical University & Shandong Academy of Medical Sciences, Jinan City (CN)

(21) Appl. No.: 17/645,501

(22) Filed: Dec. 22, 2021

(30) Foreign Application Priority Data
Mar. 9, 2021 (CN) 202110252781.3

(51) Int. Cl. G01N 33/68; G01N 1/30; G01N 27/447; G01N 30/72; G01N 30/06; G06T 7/00

(52) U.S. Cl. CPC G01N 33/6851; G01N 1/30; G01N 27/44726; G01N 30/72; G01N 30/06; G06T 7/0012; G01N 2030/027

(57) ABSTRACT

The present disclosure provides a method for identifying a human growth hormone proteoform (hGHP) pattern biomarker. The method includes: collecting an hGH-secreting pituitary adenoma tissue sample and a normal pituitary tissue sample, and extracting tissue proteins, separately; conducting two-dimensional gel electrophoresis (2DGE), western blotting, and Coomassie brilliant blue (CBB) staining, and scanning visualized polyvinylidene fluoride (PVDF) membranes and 2D gels to obtain digital images; subjecting a corresponding protein in 2D gel spot to protein digestion with trypsin and purification, and conducting mass spectrometry identification and bioinformatics analysis to identify a GHP biomarker profile; and in combination with bioinformatics, using quantitative phosphoproteomics, quantitative ubiquitinomics, and quantitative acetylomics to identify post-translational modifications (PTMs) and splicing variations in GHP. The present disclosure can identify a change pattern of GHP between a GH-secreting pituitary adenoma tissue and a normal pituitary tissue. In total, 46 GHPs are identified in the GH-secreting pituitary adenoma tissue, and only 35 GHPs are identified in the normal pituitary tissue. Therefore, 11 GHPs are only present in the GH-secreting pituitary adenoma tissue.

### CROSS REFERENCE TO RELATED APPLICATION

This patent application claims the benefit and priority of Chinese Patent Application No. 202110252781.3 filed on Mar. 9, 2021, the disclosure of which is incorporated by reference herein in its entirety as part of the present application.

### TECHNICAL FIELD

The present disclosure belongs to the technical field of molecular biology, and specifically relates to a method for identifying a human growth hormone proteoform (hGHP) pattern biomarker.

### BACKGROUND ART

Pituitary adenoma is one of the most-common primary intracranial tumors, with a prevalence rate of 17% worldwide. The abnormal growth hormone (GH) secretion occurring in pituitary adenoma can cause clinical diseases. In childhood, excessive GH secretion will cause abnormally-large bones and finally result in gigantism, but insufficient GH secretion will cause dwarfism. In adults, excessive GH secretion caused by pituitary adenoma will cause acromegaly. Pituitary adenoma causes some GH-related diseases and affects the human health over the long term. Although there are various therapeutic methods such as drug therapy and surgical resection, a series of GH-related diseases caused by pituitary adenoma still cannot get satisfactory clinical treatment. Therefore, it is one of the key methods for treating pituitary adenoma and GH-related diseases to look for GHP biomarkers for pituitary adenoma and GH-related diseases.

### SUMMARY

The technical problem to be solved by the present disclosure is to provide a method for identifying an hGHP pattern biomarker in view of the deficiencies in the art. The method can identify a change pattern of GHP between a GH-secreting pituitary adenoma tissue and a normal pituitary tissue. In total, 46 GHPs are identified in the GH-secreting pituitary adenoma tissue, and only 35 GHPs are identified in the normal pituitary tissue. Therefore, 11 GHPs are only present in the GH-secreting pituitary adenoma tissue, but not in the normal pituitary tissue.

To solve the above-mentioned technical problem, the present disclosure adopts the following technical solution: A method for identifying an hGHP pattern biomarker is provided, including:

S1. collecting an hGH-secreting pituitary adenoma tissue sample and a normal pituitary tissue sample, and lysing the tissues separately to extract two sets of tissue proteins;

S2. equally dividing each of the two sets of tissue proteins obtained in S1 into two parts, and subjecting the two parts separately to two-dimensional gel electrophoresis (2DGE) to obtain a protein-containing 2D gel a and a protein-containing 2D gel b;

S3. subjecting the protein-containing 2D gel a obtained in S2 to western blotting to obtain a visualized polyvinylidene fluoride (PVDF) membrane;

S4. soaking the protein-containing 2D gel b obtained in S2 in a Coomassie brilliant blue (CBB) staining solution to obtain a CBB-stained 2D gel b; and soaking the protein-containing 2D gel a undergoing western blotting in S3 in a CBB staining solution to obtain a CBB-stained 2D gel a;

S5. scanning the visualized PVDF membrane obtained in S3 and the CBB-stained 2D gel b and the CBB-stained 2D gel a obtained in S4 to obtain digital images; importing the digital images into Bio-Rad PDQuest 2D gel image analysis software to quantify volumes of protein spots; and matching an immuno-positive western blotting spot with corresponding protein spots in the CBB-stained 2D gel a and the CBB-stained 2D gel b;

S6. subjecting 2D gel protein spots in the CBB-stained 2D gel a and the CBB-stained 2D gel b obtained in S4 that are corresponding to an immuno-positive western blotting spot in the visualized PVDF membrane obtained in S3 to protein digestion with trypsin; and subjecting a tryptic peptide mixture to extraction and then purification with a ZipTipCis microcolumn to obtain a purified tryptic peptide mixture;

S7. subjecting the purified tryptic peptide mixture obtained in S6 to matrix-assisted laser desorption ionization time-of-flight mass spectrometry (MALDI-TOF-MS) analysis, liquid chromatography-electrospray ionization-tandem mass spectrometry (LC-ESI-MS/MS) analysis , or matrix-assisted laser desorption ionization double-time-of-flight tandem mass spectrometry (MALDI-TOF-TOF-MS/MS) analysis, to obtain peptide fingerprint (PMF) data and MS/MS data;

S8. inputting the PMF data and MS/MS data obtained in S7 into the Mascot search engine to search for proteins in the UniProt database for identification;

S9. calculating theoretical peptide masses for tryptic peptides of GH with a peptide mass tool, and aligning sequences of the tryptic peptides of GH to theoretical sequences of GH precursor, mature GH, and GH splicing variants 1, 2, 3, and 4 to determine characteristic tryptic peptides of the GH precursor, the mature GH, and the GH splicing variants 1, 2, 3, and 4; and comparing the obtained characteristic tryptic peptides with each mass spectrum obtained in S7 to determine whether a GHP is derived from the GH precursor, the mature GH, or the GH splicing variants 1, 2, 3, or 4; where the amino acid sequence of GH precursor is set forth in SEQ ID NO: 1, the amino acid sequence of mature GH is set forth in SEQ ID NO: 2, the amino acid sequence of GH splicing variant 1 is set forth in SEQ ID NO: 3, the amino acid sequence of GH splicing variant 2 is set forth in SEQ ID NO: 4, the amino acid sequence of GH splicing variant 3 is set forth in SEQ ID NO: 5, and the amino acid sequence of GH splicing variant 4 is set forth in SEQ ID NO: 6;

S10. subjecting the GH-secreting pituitary adenoma tissue and the normal pituitary tissue to quantitative phosphoproteomics: Briefly, subjecting proteins of the two tissues separately to protein digestion with trypsin, labeling a tryptic peptide mixture with an iTRAQ reagent, and enriching phosphopeptides with TiO₂; using LC-MS/MS analysis to identify an amino acid sequence and a phosphorylation site of a phosphoprotein and quantify an abundance of each phosphopeptide; and comparing an obtained tryptic peptide where a GH phosphorylation site is localized with each mass spectrum obtained in S7 to determine a phosphorylation state of a GHP;

S11. subjecting the GH-secreting pituitary adenoma tissue and the normal pituitary tissue to quantitative ubiquitinomics: Briefly, subjecting proteins of the two tissues separately to protein digestion with trypsin, and using ubiquitin antibodies to enrich ubiquitinated peptides from an obtained tryptic peptide mixture; using LC-MS/MS analysis to identify an amino acid sequence and an ubiquitination site of an ubiquitinated protein; using a label-free quantification method to quantify an abundance of an ubiquitinated peptide; and comparing an obtained tryptic peptide where a GH ubiquitination site is localized with each mass spectrum obtained in S7 to determine an ubiquitination state of a GHP; and

S12. subjecting the GH-secreting pituitary adenoma tissue and the normal pituitary tissue to quantitative acetylomics: Briefly, subjecting proteins of the two tissues separately to protein digestion with trypsin, and using acetyl antibodies to enrich acetylated peptides from an obtained tryptic peptide mixture; using LC-MS/MS analysis to identify an amino acid sequence and an acetylation site of an acetylated protein; using a label-free quantification method to quantify an abundance of an acetylated peptide; and comparing an obtained tryptic peptide where a GH acetylation site is localized with each mass spectrum obtained in S7 to determine an acetylation state of a GHP.

Compared with the prior art, the embodiments of the present disclosure have the following advantages:

1. The present disclosure can identify a change pattern of GHP between a GH-secreting pituitary adenoma tissue and a normal pituitary tissue, where there are 46 GHPs in the GH-secreting pituitary adenoma tissue and 35 GHPs in the normal pituitary tissue. The 35 GHPs in the normal pituitary tissue are matched to the 35 GHPs of the 46 GHPs in the GH-secreting pituitary adenoma tissue, but have different expression levels in the two tissues; and the remaining 11 GHPs are only present in the GH-secreting pituitary adenoma tissue, but not in the normal pituitary tissue. It demonstrates that there is a significant difference in the GHP pattern between the pituitary adenoma tissue and the normal pituitary tissue, which is most likely a biomarker profile for the abnormal expression in tumors. Different post-translational modifications (PTMs) are found in GHPs. The phosphorylation at residues Ser77, Ser132, Thr174, and Ser176 in GH is identified with phosphoproteomics; the ubiquitination at residue K96 in GH is identified with ubiquitinomics; the acetylation at residue K171 in GH is identified with acetylomics; and deamination occurs at residue Asn (N) 178 in GH.

2. The present disclosure identifies a GHP biomarker expression profile and PTMs between the GH-secreting pituitary adenoma tissue and the normal pituitary tissue, which is used to search for a GHP pattern biomarker abnormally expressed in the GH-secreting pituitary adenoma for GH-secreting pituitary adenomas and GH-related diseases. 2DGE, western blotting of 2DGE-separated proteins with anti-GH antibodies, mass spectrometry, and bioinformatics are used to identify a GHP pattern for the GH-secreting pituitary adenoma tissue and the normal pituitary tissue, respectively. In addition, quantitative phosphoproteomics, quantitative ubiquitinomics, and quantitative acetylomics are used to identify and quantify the phosphorylation, ubiquitination, and acetylation in hGHPs of the GH-secreting pituitary adenoma tissue and the normal pituitary tissue, respectively. The identified phosphorylation sites, ubiquitination sites, and acetylation sites are manually compared with mass spectra of GHPs in the GH-secreting pituitary adenoma tissue and in the normal pituitary tissue, thereby discovering the differences in GHPs and PTMs between the GH-secreting pituitary adenoma tissue and the normal pituitary tissue. It may be more conducive to discovering a GHP pattern biomarker abnormally expressed in tumors and developing corresponding diagnostic kits and targeted therapeutic drugs, which will help the early diagnosis, therapy, and prevention of GH-secreting pituitary adenomas and GH-related diseases.

The present disclosure is further described in detail below with reference to the accompanying drawings and examples.

### BRIEF DESCRIPTION OF DRAWINGS

FIG. 1 is an amino acid sequence diagram of the GH precursor and GH splicing variants 1 to 4 according to the present disclosure.

FIG. 2 shows a CBB-stained 2D gel image (a) and a 2DGE-based GH immunoaffinity western blotting image (b) for GHPs in the hGH-secreting pituitary adenoma tissue according to the present disclosure.

FIG. 3 shows a CBB-stained 2D gel image (a) and a 2DGE-based GH immunoaffinity western blotting image (b) for GHPs in the normal pituitary tissue according to the present disclosure.

FIG. 4 shows an MS/MS spectrum of the tryptic peptide LHQLAFDTYQEEFEEAYIPK (46-64) (SEQ ID NO: 7) in gel spot 36 for the hGH-secreting pituitary adenoma tissue according to the present disclosure.

FIG. 5 shows an MS/MS spectrum of the phosphorylated trypticpeptide SVFANSLVYGAS*DSNVYDLLK (121-141, S*=pSer132) (SEQ ID NO: 8) in gel spot 36 for the hGH-secreting pituitary adenoma tissue according to the present disclosure.

### DETAILED DESCRIPTION OF THE EMBODIMENTS

### EXAMPLE 1

A method for identifying an hGHP pattern biomarker was provided in this example, including:

S1. An hGH-secreting pituitary adenoma tissue sample and a normal pituitary tissue sample were collected, and the tissues were lysed separately to extract two sets of tissue proteins. A specific process was as follows:

(1) Pituitary Tissue Samples:

All hGH-secreting pituitary adenoma tissue samples (n=7) in this example came from the Department of Neurosurgery, Xiangya Hospital, and were approved by the Medical Ethics Committee of Xiangya Hospital, Central South University. Normal pituitary tissue samples (n=7) as controls were obtained from the following sources: 4 cases came from the University of Tennessee Health Science Center, and were approved by the Ethical Institution Review Committee of the University of Tennessee Medical Center; and 3 cases came from the Department of Forensic Medicine, Tongji Medical College, Huazhong University of Science and Technology, and were approved by the Tongji Medical Ethics Committee of Huazhong University of Science and Technology. These tissues were collected from surgery patients with pituitary-related diseases. The purpose and nature of the tissue collection were fully explained, and the informed consent of each patient was obtained from each patient or a family member thereof. After being taken out, these tissues were immediately frozen in liquid nitrogen and then stored at −80° C. for later use.

(2) Protein Extraction:

The pituitary adenoma tissue samples and normal pituitary tissue samples were taken out from −80° C. freezer and slowly thawed at room temperature. The tissue sample (approximately 500 mg) was washed with 0.9% NaCl (5 mL, 5×) to remove blood on the surface of the tissue, and then fully cut with clean scissors into very small pieces (approximately 1 mm³, on ice). A volume (4 mL) of protein extraction buffer was added to the tissue pieces, and a resulting mixture was thoroughly mixed and subjected to lysis on ice for 2 h. The protein extraction buffer included: 7 mol/L urea, 2 mol/L thiourea, 60 mmol/L dithiothreitol (DTT), 4% (w/v) 3-[(3-cholamidopropyl)dimethylammonio]-1-propane sulphate (CHAPS), 0.5% v/v immobilized pH gradient (IPG) buffers (which were added just prior to use), and a trace of bromophenol blue (BPB). Then the resulting extraction solution containing proteins was centrifuged (15,000×g, 15 min, 4° C.), and a resulting supernatant was collected as a protein sample solution.

S2. Each of the two sets of tissue proteins obtained in S1 was equally divided into two parts, and the two parts were separately subjected to 2DGE to obtain a protein-containing 2D gel a and a protein-containing 2D gel b. A specific process was as follows:

(3) 2DGE and Western Blotting:

3.1. First-Dimension Isoelectric Focusing Polyacrylamide Gel Electrophoresis (IEF-PAGE)

IEF-PAGE was conducted on the IPGphor IEF system. A prepared pituitary adenoma protein sample or control protein sample (500 μg) was loaded on an 18-cm IPG strip (avoiding bubbles), and 3 mL of mineral oil was added to cover the IPG strip. Rehydration was conducted for about 18 h. IEF-PAGE was conducted at room temperature. Parameters were as follows: each IPG strip had a fixed maximum current of 30 μA and a temperature of 20° C.; step 1: constant 250 V, 1 h, 125 Vh; step 2: gradient 1,000 V, 1 h, 500 Vh; step 3: gradient 8,000 V, 1 h, 4,000 Vh; step 4: 8,000 V, 4 h, 32,000 Vh; and step 5: constant 500 V, 0.5 h, 250 Vh. Finally, the IEF involved a total time of 7.5 h and a total voltage-time product of 36,875 Vh. After the IEF, the IPG strip was taken out, and the mineral oil was removed from the plastic back of the IPG strip.

3.2 Second-Dimension Sodium Dodecyl Sulfate Polyacrylamide Gel Electrophoresis (SDS-PAGE).

A vertical electrophoresis system was used to subject IEF-separated proteins in an IPG strip to SDS-PAGE separation. A 12% PAGE separation gel (n=12) was prepared as follows: 180 mL of 40% (w/v) acrylamide/bis-acrylamide stock solution (29:1, w:w), 150 mL of 1.5 mol/L Tris-HCl (pH 8.8), 3 mL of 10% ammonium persulfate (AP), 270 mL of ddH₂O (DD water), and 150 μL of tetramethylethylenediamine (TEMED) were mixed to prepare the 12% PAGE separation gel (n=12); and a resulting mixed solution was slowly poured into a gel-forming glass frame until the solution was 1 cm away from an upper end of the glass frame, about 3 mL of DD water was immediately added to cover an upper end of the gel, and a resulting product was placed at room temperature for 1 h. An IPG strip with IEF-separated proteins was taken out from an IEF instrument, and shaken gently for about 15 min in 25 mL of reduction equilibration buffer. The reduction equilibration buffer was obtained by mixing 375 mmol/L Tris-HCl (pH 8.8), 20% v/v glycerol, 2% W/v SDS, 6 mol/L urea, a trace of BPB, and 2% (w/v) DTT. Then the IPG strip was shaken gently for 15 min in 25 mL of alkylation equilibrium buffer. The alkylation equilibrium buffer was composed of 375 mmol/L Tris-HCl (pH 8.8), 20% v/v glycerol, 2% w/v SDS, 6 mol/L urea, a trace of BPB, and 2.5% w/v iodoacetamide (IAA) (which was added just prior to use). Each equilibrated IPG strip was placed on a top of the 12% PAGE separation SDS-PAGE gel, and 30 mL of boiling SDS electrophoresis buffer with 1% agarose was quickly poured to cover the SDS-PAGE resolving gel (avoiding bubbles in the gel); the IPG strip with the IEF-separated proteins was then evenly and quickly pushed into the upper agarose solution; and after the agarose solution solidified, the SDS-PAGE resoling gel plate was transferred to an electrophoresis tank with 10 L of electrophoresis buffer (composed of 25 mmol/L Tris, 192 mmol/L glycerol, and 0.1% w/v SDS), and then electrophoresis was conducted at 200 V for about 370 min

S3. The protein-containing 2D gel a obtained in S2 was subjected to western blotting with anti-GH antibody to obtain a visualized PVDF membrane. A specific process was as follows:

(4) 2DGE-Based Western Blotting:

After the electrophoresis, the 2D gel between two glass plates was taken out, and a small piece was cut off at a negative end of the upper left corner to mark a direction of the 2D gel electrophoresis. Proteins in the 2D gel were transferred to a PVDF membrane with an Amersham Multiphor-II semi-dry electrotransfer system. Specific steps were as follows: an anode electrode plate was placed in a buffer tank of an electrotransfer tank, and then the anode electrode plate was allowed to be saturated with DD water; 6 sheets of filter paper were soaked in an anode transfer buffer R until an equilibrium was reached, and then placed on the anode plate; 3 sheets of filter paper were soaked in a transfer buffer T until an equilibrium was reached, and then placed on the 6 sheets of filter paper; a PVDF membrane was soaked in the anode transfer buffer until an equilibrium was reached, and then placed on the 3 sheets of filter paper; then the 2D gel was placed on the PVDF membrane; 9 sheets of filter paper were soaked in a transfer buffer S until an equilibrium was reached, and then placed on the 2D gel; and electrotransfer was conducted for 90 min at a constant current of 0.8 mA/cm². A PVDF membrane bound with proteins was soaked in 100 mL of BSA/PBST buffer prepared from Tween-20 and 0.3% bovine serum albumin (BSA)/phosphate buffer solution (PBS) to block for 60 min (gently shaking, room temperature). After the blocking was completed, the PVDF membrane was washed 3 times with DD water. Proteins bound to the PVDF membrane were incubated in 100 mL of primary antibody diluent containing 100 μL of rabbit anti-GH antibody for 1 h (slightly shaking, room temperature), and washed 4 times with 200 mL of PBST solution (each 15 min) and then 2 times with DD water. The proteins on the PVDF membrane were incubated in 20 pL of secondary antibody (goat anti-rabbit alkaline phosphatase-conjugated IgG) diluted with 100 mL of 0.3% BSA/PBST, and washed 3 times with 200 mL of PBST solution (each 15 min) and then 3 times with DD water. The PVDF membrane was stained with 1-Step nitro blue tetrazolium/5-bromo-4-chloro-3-indolyl phosphate (NBT/BCIP) (Thermo Product, No. 3404) to visualize the proteins on the PVDF membrane. Moreover, a parallel negative control experiment (without the primary antibody anti-hGH antibody) was conducted to detect whether the secondary antibody undergoes a cross reaction. The PVDF membrane with the visualized proteins was dried and stored between two filter papers.

S4. The protein-containing 2D gel b obtained in S2 was soaked in a CBB staining solution to obtain a CBB-stained 2D gel b; and the protein-containing 2D gel a undergoing western blotting in S3 was soaked in a CBBstaining solution to obtain a CBB-stained 2D gel a.

S5. The visualized PVDF membrane obtained in S3, and the CBB-stained 2D gel b and the CBB-stained 2D gel a obtained in S4 were scanned to obtain digital images; the digital images were imported into Bio-Rad PDQuest 2D gel image analysis software to quantify volumes of protein spots; and an immuno-positive western blotting spot was matched with corresponding protein spots in the CBB-stained 2D gel a and the CBB-stained 2D gel b. A specific process was as follows:

(5) Protein Staining and Image Analysis of the 2D Gel: (Yellow Labels Indicate a or b, or a and b)

The protein-containing 2D gels a and b were soaked in a CBB staining solution for about 2 h to 3 h (slowly shaking), and then washed with DD water twice, with 20% v/v absolute ethanol until the gel background faded to be almost colorless (gently shaking), and finally with DD water twice. The CBB staining solution was composed of 0.75 g of CBB G250, 30 mL of glacial acetic acid, 135 mL of methanol, and 30 mL of DD water. The visualized PVDF membrane and the corresponding CBB-stained 2D gels a and b were scanned to obtain digital images. The digital images were imported into Bio-Rad PDQuest 2D gel image analysis software (version 7.0) to quantify volumes of protein spots; and an immuno-positive western blotting spot was matched with corresponding 2D gel protein spots in the CBB-stained 2D gels a and b.

S6. 2D gel protein spots in the CBB-stained 2D gels a and b obtained in S4 that were corresponding to an immuno-positive western blotting spot in the visualized PVDF membrane obtained in S3 were subjected to protein digestion with trypsin; and a tryptic peptide mixture was subjected to extraction and then purification with a ZipTipCis microcolumn to obtain a purified tryptic peptide mixture.

S7. The purified tryptic peptide mixture obtained in S6 was subjected to MALDI-TOF-MS analysis to obtain PMF data, LC-ESI-MS/MS analysis to obtain MS/MS data, or MALDI-TOF-TOF-MS/MS analysis to obtain PMF and MS/MS data.

S8. PMF data and MS/MS data obtained in S7 were input into the Mascot search engine to search for proteins in the UniProt database for identification. A specific process was as follows:

(6) Mass Spectrometry Identification of hGH:

2D gel protein spots corresponding to immuno-positive western blotting spots were cut off and subjected to protein digestion with trypsin, and a tryptic peptide mixture was subjected to purification with the ZipTipC₁₈ microcolumn. The purified tryptic peptide mixture was analyzed with the following three mass spectrometry methods: MALDI-TOF-MS, LC-ESI-MS/MS, or MALDI-TOF-TOF-MS/MS.

For MALDI-TOF-MS analysis, the purified tryptic peptide mixture was mixed with an a-cyano-4-hydroxycinnamic acid (CHCA) matrix and analyzed with the MALDI-TOF Voyager DE-RP mass spectrometer (Framingham, MA, USA) to obtain PMF data; and the PMF data were input into the Mascot search engine to query the UniProt database 91215 (date: Jul. 2, 2019; 513,877 sequences; 180,750,753 residues; and 513,877 human sequences) to identify proteins. In addition, a blank control experiment was conducted, and the margin gel pieces were analyzed with MALDI-TOF-MS to eliminate the contaminant mass ion peaks derived from contaminants such as keratin and trypsin.

For LC-ESI-MS/MS analysis, the purified tryptic peptide mixture was analyzed with LC-ESI-Q-IT (quadrupole ion trap mass spectrometer, Thermo Finnigan, San Jose, Calif., USA) to obtain MS/MS data. Instrument parameters were set as follows: capillary probe temperature: 110° C., electrospray ionization mass spectrometer voltage: 2 KV, and electronic multiplier voltage: −900 V. The MS/MS data were input into MASCOT software to search in the UniProt and NCBInr human databases for protein identification.

For the MALDI-TOF-TOF-MS/MS analysis, the purified tryptic peptide mixture was mixed with a CHCA matrix and then analyzed with a MALDI-TOF-TOF mass spectrometer to obtain PMF and MS/MS data. The parameters were set as follows: reflection mode, acceleration voltage: 25 kV, and scanning range (m/z): 800 to 4,000. MS and MS/MS data were commonly input into the MASCOT software for protein identification in the UniProt human protein database. In this study, all MASCOT searches got a score of over 70. The score of 70 was a statistical threshold for the identity or high-degree homology between a searched sequence and an identified sequence, which was statistically significant (P<0.05).

The amino acid sequence of hGH came from the UniProt protein database (www.expasy.ch). In order to accurately and reliably identify hGH in hGH-secreting pituitary adenoma tissues and control normal pituitary tissues with mass spectrometry, common ion mass peaks introduced by the blank gels should be removed from mass spectra before searching against the protein database. Because these blank gels included common contaminants such as trypsin, keratin from skin and hair, matrix, and other unknown contaminants, which would produce ion mass peaks. Ion mass peaks of these contaminants usually have the following m/z values: 842.5, 870.5, 1045.4, 1109.3, 1179.3, 1235.2, 1277.4, 1307.3, 1365.3, 1383.3, 1434.4, 1475.3, 1493.3, 1638.3, 1708.2, 1716.3, 1791.1, 1838.3, 1940.2, 1994.2, 2211.1, 2225.1, 2239.1, 2284.1, 2389.8, 2705.7, and 2871.9.

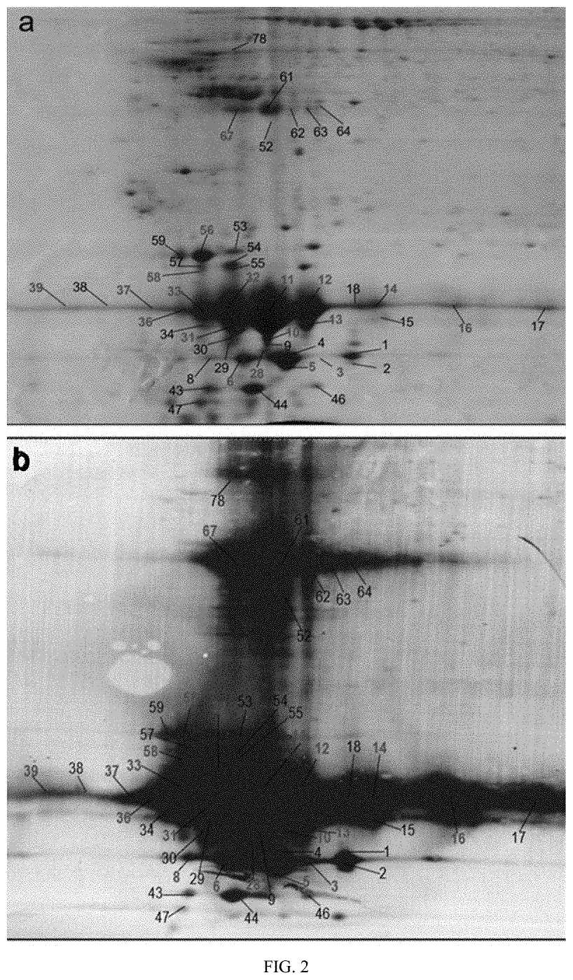

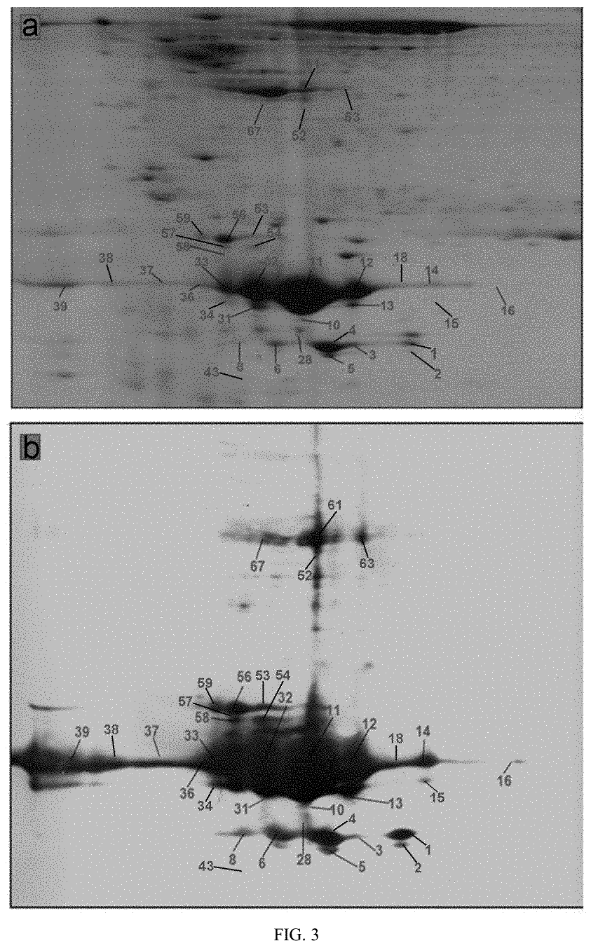

Therefore, through comparative analysis of the 2DGE-based western blotting image and the corresponding CBB image of 2DGE, 46 GH immuno-positive spots were identified in the GH-secreting pituitary adenoma tissue (FIG. 2a and FIGS. 2b), and 35 GH immuno-positive spots were identified in the normal pituitary tissue (FIG. 3a and FIG. 3b). Moreover, the 35 GH immuno-positive spots in the normal pituitary were matched with 35 among the 46 spots in the GH-secreting pituitary adenoma tissue, and the remaining 11 GH immuno-positive spots were only present in the GH-secreting pituitary adenoma tissue, but not in the normal pituitary tissue. In addition, the Bio-Rad PDQuest 2D gel image analysis was used to quantify volumes of CBB-stained protein spots corresponding to the 46 GH immuno-positive spots in the GH-secreting pituitary adenoma tissue (FIG. 2a) and volumes of CBB-stained protein spots corresponding to the 35 GH immuno-positive spots in the normal pituitary tissue (FIG. 3a); GHPs 9, 17, 29, 30, 44, 46, 47, 55, 62, 64, and 78 (n=11) were only present in the GH-secreting pituitary adenoma tissue; and compared to the normal pituitary tissue, GHPs 3, 38, 39, 52, and 63 (n=5) showed a reduced abundance in the GH-secreting pituitary adenoma tissue, while GHPs 1, 2, 4, 5, 6, 8, 10, 11, 12, 13, 14, 15, 16, 18, 28, 31, 32, 33, 34, 36, 37, 43, 53, 54, 56, 57, 58, 59, 61, and 67 (n =30) showed an increased abundance in the GH-secreting pituitary adenoma tissue.

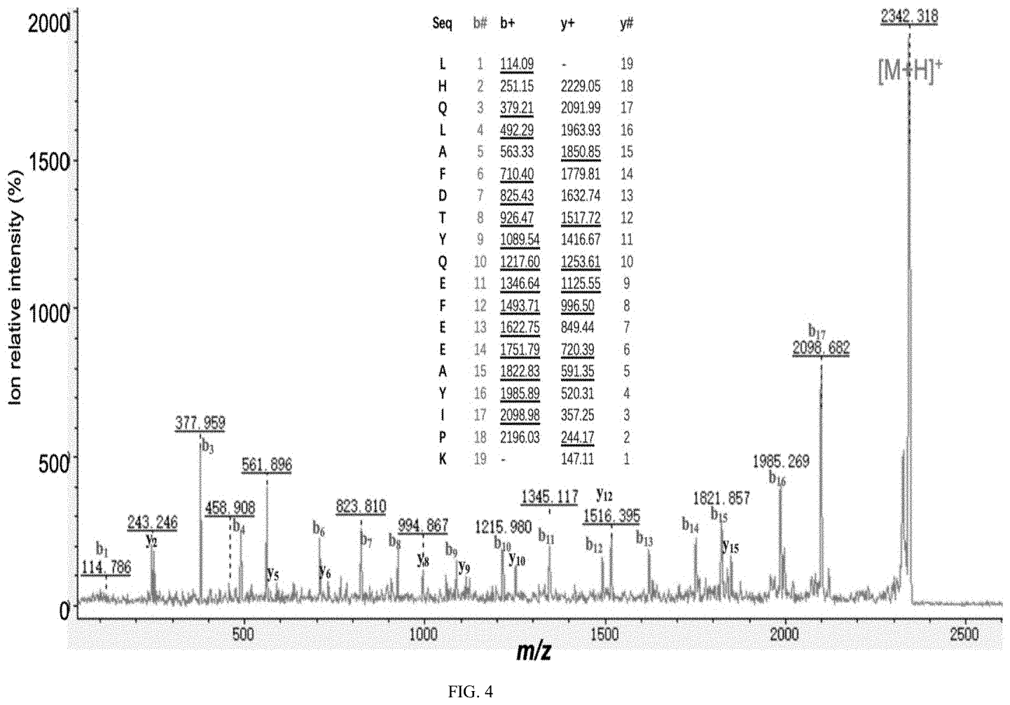

The protein in each cut gel spot was subjected to protein digestion with trypsin, and then tryptic peptides were extracted and purified. The prepared tryptic peptide mixture was analyzed with MALDI-TOF-TOF-MS to obtain PMF data or MS/MS data, and then the human protein database was searched to conduct protein identification. For the GH-secreting pituitary adenoma tissue (FIG. 2a), mass spectrometry analysis showed that the 46 2D gel spots all included hGH (UniProt: P01241). For the normal pituitary tissue (FIG. 3a), among the 35 2D gel spots, mass spectrometry analysis showed that 25 2D gel spots included hGH (UniProt: P01241); and no protein was identified with mass spectrometry in 2D gel spots 2, 15, 18, 38, 52, 53, 54, 57, 61, and 63 (n=10), but the 10 spots (FIG. 3a) had a positive GH immunoaffinity image (FIG. 3b), and the corresponding 2D gel spots for the GH-secreting pituitary adenoma tissue included hGH (FIG. 2a). The gel spot 36 of the GH-secreting pituitary adenoma tissue was taken as an example to illustrate the mass spectrometry identification of GH (FIG. 4), and the figure showed an MS/MS mass spectrum of the GH-derived tryptic peptide LHQLAFDTYQEFEEAYIPK (46-64) (SEQ ID NO: 7) in the gel spot 36 for the hGH-secreting pituitary adenoma tissue.

S9. Theoretical peptide masses for tryptic peptides of GH were calculated with a peptide mass tool, and sequences of the tryptic peptides of GH were aligned to theoretical sequences of GH precursor, mature GH, and GH splicing variants 1, 2, 3, and 4 to determine characteristic tryptic peptides of the GH precursor, the mature GH, and the GH splicing variants 1, 2, 3, and 4; and the obtained characteristic tryptic peptides were compared to each mass spectrum obtained in S7 to determine whether a GHP is derived from the GH precursor, the mature GH, or the GH splicing variants 1, 2, 3, or 4. A specific process was as follows:

(7) Identification of hGH Splicing Variants:

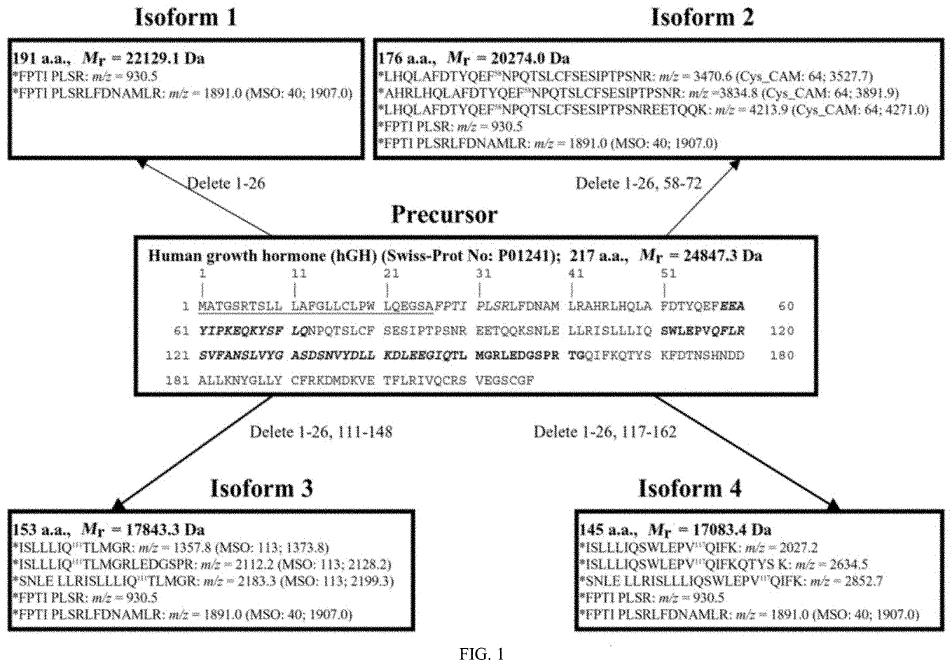

As shown in FIG. 1, the tryptic peptides of the GH precursor (Precursor in FIG. 1, SEQ ID NO: 1) and the mature GH (Isoform 1 in FIG. 1, SEQ ID NO: 2), and the GH splicing variants 1, 2, 3, and 4 (Isoform 1 to 4 in FIG. 1, SEQ ID NOs: 3-6) had different characteristic amino acid sequences, which were easy to be identified according to mass spectrum peaks. The peptide mass tool (http://us.expasy.org/cgi-bin/peptide-mass.pl) was used to calculate the theoretical mass values of the tryptic peptides of the GH precursor and mature GH, and the GH splicing variants 1, 2, 3, and 4. Trypsin digestion parameters were set as follows: the trypsin cleavage site was located at the C termini of Lys (K) and Arg (R); the maximum missed cleavage number was 2; all reduced cysteine and oxidized methionine were involved; the peptide mass was greater than 500 Da; the peptide amino acid sequence used the monoisotopic mass; and the peptide ion was set to [M+H]⁺. These parameters were consistent with the parameters of MALDI-TOF or MALDI-TOF-TOF PMF data analysis, which would distinguish the characteristic tryptic peptides of the GH precursor, mature GH, and GH splicing variants 1, 2, 3, and 4. These characteristic tryptic peptides were compared to each GH PMF to determine a splicing variation state of GH in the GH-secreting pituitary adenoma tissue and the control pituitary tissue.

Results showed that, except for GHP 46 in the GH-secreting pituitary adenoma, the characteristic tryptic peptide ion FPTIPLSR (position 27-34, [M+H]⁺, m/z=930.5) (SEQ ID NO: 9) appeared in all PMFs of GHPs for the normal pituitary tissue and GH-secreting pituitary adenoma tissue, demonstrating that the signal peptide was removed from these GHPs (positions 1-26, underlined in FIG. 1) (SEQ ID NO: 10). For GHP 46 in the GH-secreting pituitary adenoma tissue, the PMF did not include the characteristic tryptic peptide ion FPTIPLSR (position 27-34, [M+H]⁺, m/z=930.5) (SEQ ID NO: 9), but included the characteristic tryptic peptide ion TSLLLAFGLLCLPWLQEGSAFPTIPLSR (position 7-34, [M+H]⁺, m/z=3043.7) (SEQ ID NO: 11), which clearly showed that the GHP 46 included a signal peptide. Moreover, for the GH-secreting pituitary adenoma tissue, GHPs 1 and 5 were the splicing variant 2, GHP 78 was the splicing variant 3, and the remaining 43 GHPs were the splicing variant 1, where the splicing variant 4 was not discovered; and for the normal pituitary tissue, GHPs 3, 4, and 6 were the splicing variant 2, and the remaining GHPs were the splicing variant 1, where the splicing variants 3 and 4 were not discovered.

S10. The GH-secreting pituitary adenoma tissue and the normal pituitary tissue were subjected to quantitative phosphoproteomics: Briefly, proteins of the two tissues were separately subjected to protein digestion with trypsin, a tryptic peptide mixture was labeled with an iTRAQ reagent, and phosphopeptides were enriched with TiO₂; LC-MS/MS analysis was used to identify an amino acid sequence and a phosphorylation site of a phosphoprotein and quantify an abundance of each phosphopeptide; and an obtained tryptic peptide where a GH phosphorylation site was localized was compared to each mass spectrum obtained in S7 to determine a phosphorylation state of GHP.

S11. The GH-secreting pituitary adenoma tissue and the normal pituitary tissue were subjected to quantitative ubiquitinomics: Briefly, proteins of the two tissues were separately subjected to protein digestion with trypsin, and ubiquitin antibodies were used to enrich ubiquitinated peptides from an obtained tryptic peptide mixture; LC-MS/MS analysis was used to identify an amino acid sequence and an ubiquitination site of an ubiquitinated protein; a label-free quantification method was used to quantify an abundance of an ubiquitinated peptide; and an obtained tryptic peptide where a GH ubiquitination site was located was compared to each mass spectrum obtained in S7 to determine an ubiquitination state of GHP.

S12. The GH-secreting pituitary adenoma tissue and the normal pituitary tissue were subjected to quantitative acetylomics: Briefly, proteins of the two tissues were separately subjected to protein digestion with trypsin, and acetyl antibodies were used to enrich acetylated peptides from an obtained tryptic peptide mixture; LC-MS/MS analysis was used to identify an amino acid sequence and an acetylation site of an acetylated protein; a label-free quantification method was used to quantify an abundance of an acetylated peptide; and an obtained tryptic peptide where a GH acetylation site was located was compared to each mass spectrum obtained in S7 to determine an acetylation state of GHP.

A specific process was as follows:

(8) PTM State of hGHP:

The hGH-secreting pituitary adenoma tissue and the control tissue were each subjected to quantitative phosphoproteomics (enriching phosphopeptides with TiO₂), quantitative ubiquitinomics (enriching ubiquitinated peptides with ubiquitin antibodies), and quantitative acetylomics (enriching acetylated peptides with acetyl antibodies).

8.1. For phosphoproteomics analysis, the 6-plex iTRAQ kit was used to label tryptic peptides of the hGH-secreting pituitary adenoma tissue sample (the labeling was repeated 3 times) and the control normal pituitary tissue sample (the labeling was repeated 3 times). Basic steps were as follows: An amount (200 μg) of proteins was subjected to protein digestion with trypsin, desalination with C₁₈, and vacuum centrifugal concentration, and then the absorbance at 280 nm on the UV spectrum was used for quantification; a tryptic peptide mixture (100 μg) of each sample was labeled with an iTRAQ reagent, and iTRAQ-labeled peptides were mixed equally (1:1:1:1:1:1), concentrated by a vacuum concentrator, and then diluted in 500 μl of DHB buffer; TiO₂ beads were added, and a resulting mixture was stirred gently for 2 h and then centrifuged (5,000×g; 1 min); phosphopeptide-containing beads were collected and washed 3 times with 50 μl of washing buffer I (30% ACN and 3% TFA) and 50 μl of washing buffer II (80% ACN and 0.3% TFA) to obtain phosphopeptide beads; and enriched phosphopeptides were eluted 3 times with 50 μl of elution buffer (40% ACN and 15% NH₄OH), and lyophilized, and subjected to LC-MS/MS analysis to obtain MS/MS data. The human protein database was queried with the MS/MS data to identify an amino acid sequence and a phosphorylation site of a phosphoprotein, and the intensity of the iTRAQ reporter ion was used to quantify the abundance of each phosphopeptide.

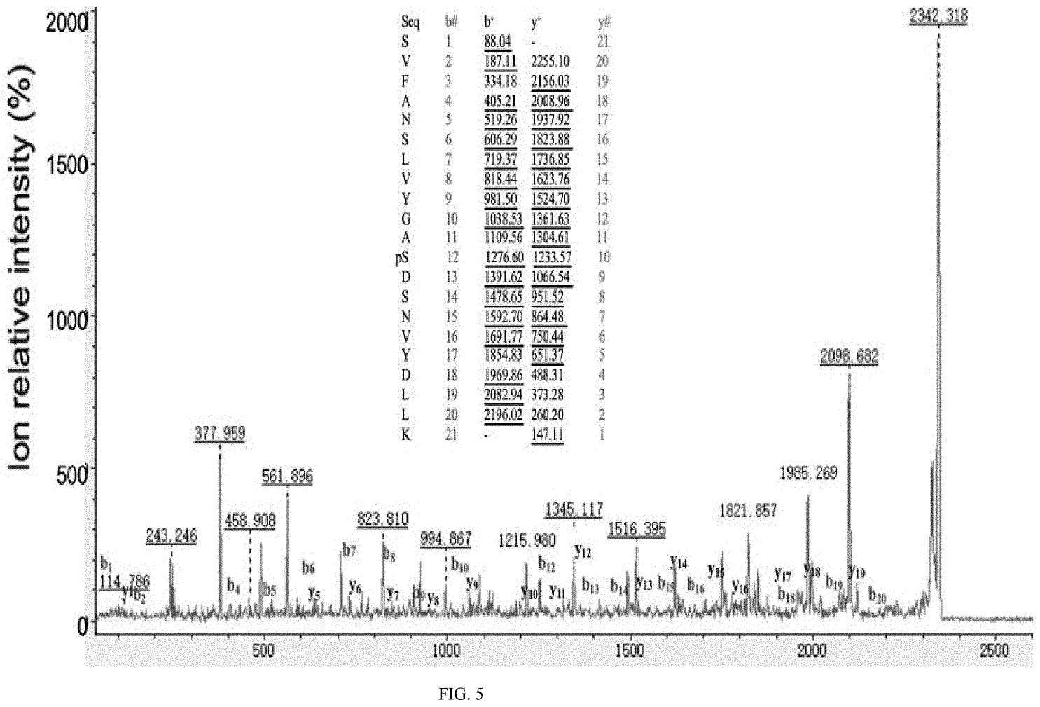

Results showed that phosphorylation at four residues Ser132, Ser134, Thr174, and Ser176 in hGH (P02141) were identified with quantitative phosphoproteomics, and the tryptic peptide ions SVFANSLVYGASDSNVYDLLK(121-141) (SEQ ID NO: 8), FDTNSHNDDALLK (172-184) (SEQ ID NO: 12), QTYSKFDTNSHNDDALLK (167-184) (SEQ ID NO: 13), and FDTNSHNDDALLKNYGLLYCFR (172-193) (SEQ ID NO: 14) that the four phosphorylation sites were located and masses of corresponding phosphopeptides were theoretically calculated. Masses of these phosphopeptides were compared with PMF of each GHP to determine a phosphorylation state of the GHP. The tryptic peptide SVFANSLVYGASDSNVYDLLK (121-141) (SEQ ID NO: 8) had a [M+H]⁺ ion at m/z=2262.1; and if Ser132 is phosphorylated, the tryptic peptide will have a [M+H]⁺ ion at m/z=2342.1. However, there was another tryptic peptide ion [M+H]⁺ (LHQLAFDTYQEFEEAYIPK, 46-64) (SEQ ID NO: 7) in the tryptic peptides of hGH, which was also at m/z=2342.1. The amino acid sequences of the peptides ([M+H]⁺, m/z=2342.3) were determined with MS/MS analysis (FIG. 4 to FIG. 5), and the amino acid sequences of the two peptides LHQLAFDTYQEFEEAYIPK (46-64, FIG. 4) (SEQ ID NO: 7) and SVFANSLVYGAS*DSNVYDLLK (121-141, S*=phosphorylated Ser132, FIG. 5) (SEQ ID NO: 8) were both identified. In the normal pituitary tissue, only 4 GHPs (11, 32, 33, and 56) had the phosphorylation of Ser132; and in the GH-secreting pituitary adenoma tissue, 3 GHPs (12, 36, and 54) had the phosphorylation at residue Ser132, 9 GHPs (9, 10, 13, 16, 17, 33, 38, 43, and 44) had the phosphorylation at residue Ser77, and 3 GHPs (6, 39, and 78) had the phosphorylation at residue Thr174 or Ser176.

8.2. For the acetylomics analysis, proteins in the pituitary adenoma tissue and the control pituitary tissue were each subjected to protein digestion with trypsin, and each tryptic peptide mixture was incubated with anti-N-acetyl lysine antibody beads for 2 h; and a resulting mixture was centrifuged (1 min, 4° C., 1000 g), and a resulting supernatant was discarded. The anti-N-acetyl lysine antibody beads with acetylated peptides were washed to remove non-specific binding peptides, and then the acetylated peptides were eluted with 40 μL of 0.1% TFA solution, desalinated with C₁₈ STAGE Tips, and analyzed with LC-MS/MS to obtain MS/MS data. LC was conducted with a reversed-phase trap column (Thermo Scientific Acclaim PepMap100, 100 μm×2 cm, nanoViper C 18) and a C₁₈ reversed-phase analytical column (Thermo Scientific Easy Colum: length: 10 cm, inner diameter: 75 μm, and resin: 3 μm), and during gradient elution, a sample passed through a buffer A (0.1% formic acid) and a buffer B (84% acetonitrile and 0.1% formic acid) at a flow rate of 300 nL/min for 120 min. The LC linear gradient was set as follows: linear gradient of solution B: 0% to 55% at 0 min to 90 min, 55% to 100% at 90 min to 105 min, and 100% 105 min to 120 min. MS/MS parameters of the Q-Exactive mass spectrometer were set as follows: positive ion mode; precursor ion scanning range (m/z): 300 to 1800; automatic gain control (AGC) target: 3e6; and at m/z=200, MS scanning resolution: 70,000, and MS/MS scanning resolution: 17,500. MS/MS data were input into the MaxQuant software to identify an amino acid sequence and an acetylation site of an acetylated protein, and a label-free quantification method was used to quantify the abundance of an acetylated peptide.

Results showed that an acetylation site was identified at residue Lys171 (K171) in hGH with quantitative acetylomics, and the mass of residue K171 increased by 42 Da. In addition, a protein modification software predicted that residues Lys64, 96, 141, 166, 171, 194, and 198 in hGH (P01241) were potential acetylation sites, which further confirmed the accuracy of K171 acetylation in hGH (P012141) identified with acetylomics. The ion masses of the acetylated peptides with the acetylation site K171 were theoretically calculated, thus 6 theoretical acetylated peptides were obtianed, including TGQIFKQTYSK (161-171) (SEQ ID NO: 15), QTYSK (167-171) (SEQ ID NO: 16) , LEDGSPRTGQIFKQTYSK (154-171) (SEQ ID NO: 17), QTYSKFDTNSHNDDALLK (167-184) (SEQ ID NO: 13), TGQIFKQTYSKFDTNSHNDDALLK (161-184) (SEQ ID NO: 18), and QTYSKFDTNSHNDDALLKNYGLLYCFR (167-193) (SEQ ID NO: 19). Then, these 6 theoretical acetylated peptides were compared to the PMF of each GHP to determine an acetylation state of the hGHP, and it was found that only the tryptic peptide QTYSK*FDTNSHNDDALLKNYGLLYCFR (167-193, K*=ac-Lys171) (SEQ ID NO: 19) in GHP in spot T30 from the GH-secreting pituitary adenoma tissue was acetylated.

8.3. For the ubiquitinomics analysis, proteins in the hGH-secreting pituitary adenoma tissue sample and the control normal pituitary tissue sample were each subjected to protein digestion with trypsin; a tryptic peptide mixture was incubated with anti-K-c-GG antibody beads [PTMScan Ubiquitin Remnant Motif (K-ε-GG) kit], and then the beads were washed and centrifuged to remove non-specific binding peptides; the anti-K-ε-GG antibody beads with ubiquitinated peptides were subjected to elution in 40 μL of 0.15% TFA solution, and an eluate was subjected to desalination with C₁₈ STAGE and then to LC-MS/MS analysis to obtain MS/MS data. MS/MS data were input into the MaxQuant software to identify an amino acid sequence and an ubiquitination site of an ubiquitinated protein, and a label-free quantification method was used to quantify the abundance of an ubiquitinated peptide.

Results showed that an ubiquitination site was identified at residue Lys96 (K96) in hGH with quantitative ubiquitinomics, and the mass of the residue increased by 114 Da. According to theoretical calculations, there were 4 ubiquitinated tryptic peptides with the ubiquitination at residue K96 in hGH (P01241), including YSFLQNPQTSLCFSESIPTPSNREETQQK (68-96) (SEQ ID NO: 20), EETQQKSNLELLR (91-103) (SEQ ID NO: 21), EETQQK (91-96) (SEQ ID NO: 22), and EETQQKSNLELLRISLLLIQSWLEPVQFLR (91-120) (SEQ ID NO: 23). Then, these 4 theoretical ubiquitinated peptides were compared to the PMF of each GHP to determine a ubiquitination state of the hGHP, and it was found that only the tryptic peptide EETQQK*SNLELLR (91-103; K*=ub-Lys96) (SEQ ID NO: 21) was ubiquitinated in GHP in spot T78.

8.4. For the protein deamination analysis, the deamination of glutamine (Q) and asparagine (N) residues led to the formation of corresponding glutamic acid (E) and aspartic acid (D), which was accompanied by a mass increase of 1 Da and an apparent pI decrease to pH=7.4. Carboxylic anions are usually derived from protein aging, and may also be derived from the basic conditions for storing protein samples. Deamination usually occurs in 2D gels, which will cause a protein to undergo a series of different pI values with a similar Mᵣ. In this study, deamination at the residue Asn178 (D178) was detected in 25 GHPs (1, 5, 6, 8, 9, 13, 14, 16, 17, 18, 28, 30, 34, 36, 38, 39, 43, 44, 53, 54, 57, 58, 59, 63, and 67) in the GH-secreting pituitary adenoma tissue, and in 5 GHPs (1, 14, 31, 32, and 56) in the normal pituitary tissue.

In summary, with the analysis of 2DGE-based western blotting maps, the corresponding CBB 2DGE maps, MALDI-TOF-MS-PMF, LC-ESI-MS/MS, and MALDI-TOF-TOF-MS/MS data, quantitative phosphoproteomics, ubiquitinomics, and acetylomics, deamination analysis, and data processing, it was found that there were 46 GHPs in the GH-secreting pituitary adenoma tissue, and 35 GHPs in the normal pituitary tissue; and 11 among the 46 GHPs were present only in the GH-secreting pituitary adenoma tissue, but not in the normal pituitary tissue. This abnormal GHP change pattern is most likely a biomarker for the abnormal expression in tumors. Further, the phosphorylation, ubiquitination, acetylation, and deamination modification states of GHPs were identified between the pituitary adenoma tissue and the normal pituitary tissue, i. e., the differential modification between the tumor and the control were identified.

In this example, 46 GHPs were identified in the GH-secreting pituitary adenoma tissue, and 35 GHPs were identified in the normal pituitary tissue;

35 among the 46 GHPs in the GH-secreting pituitary adenoma tissue were matched with the 35 GHPs in the control pituitary tissue, but showed different expression levels; and

the remaining 11 of the 46 GHPs were only present in the GH-secreting pituitary adenoma tissue, but not in the normal pituitary tissue.

It indicates that the GHP change pattern is most likely a biomarker for the abnormal expression in tumors.

In the GH-secreting pituitary adenoma tissue, the GHP in gel spot 46 was a GH precursor, and the GHPs in the remaining spots were all mature GH.

In the GH-secreting pituitary adenoma tissue, GHPs (1 and 5) were the splicing variant 2, GHP 78 was the splicing variant 3, and the remaining 43 GHPs were the splicing variant 1, where the splicing variant 4 was not discovered; and in the normal pituitary tissue, GHPs (3, 4, and 6) were the splicing variant 2, and the remaining GHPs were the splicing variant 1, where the splicing variants 3 and 4 were not discovered. [82] The amino acid sequences of the two tryptic peptides LHQLAFDTYQEFEEAYIPK (46-64, FIG. 4) (SEQ ID NO: 7) and SVFANSLVYGAS*DSNVYDLLK (121-141, S*=phosphorylated Ser132, FIG. 5) (SEQ ID NO: 8) were determined with the MS/MS Data. In the GH-secreting pituitary adenoma tissue, 3 GHPs (12, 36, and 54) had the phosphorylation at residue Ser132, 9 GHPs (9, 10, 13, 16, 17, 33, 38, 43, and 44) had the phosphorylation at residue Ser77, and 3 GHPs (6, 39, and 78) had the phosphorylation at residue Thr174 or Ser176; and in the normal pituitary tissue, 4 GHPs (11, 32, 33, and 56) had the phosphorylation at residue Ser132. These data clearly proved the difference in phosphorylation of GHPs between the GH-secreting pituitary adenoma tissue and the normal pituitary tissue.

In the GH-secreting pituitary adenoma tissue, the tryptic peptide EETQQK*SNLELLR (91-103, K* =Ub-Lys96) (SEQ ID NO: 21) of GHP in T78 was ubiquitinated, which was not ubiquitinated in the control pituitary tissue. These data clearly proved the difference in ubiquitination of GHPs between the GH-secreting pituitary adenoma tissue and the normal pituitary tissue.

In the GH-secreting pituitary adenoma tissue, the tryptic peptide QTYSK*FDTNSHNDDALLKNYGLLYCFR (167-193, K*=acetylated Lys171) (SEQ ID NO: 19) of GHP in T30 was acetylated, which was not acetylated in the control pituitary tissue. These data clearly proved the difference in acetylation of GHPs between the GH-secreting pituitary adenoma tissue and the normal pituitary tissue.

In the GH-secreting pituitary adenoma tissue, deamination at residue Asn178 (D178) was detected in 25 GHPs (1, 5, 6, 8, 9, 13, 14, 16, 17, 18, 28, 30, 34, 36, 38, 39, 43, 44, 53, 54, 57, 58, 59, 63, and 67); and in the normal pituitary tissue, deamination at residue Asn178 (D178) was detected in 5 GHPs (1, 14, 31, 32, and 56). These data clearly proved the difference in deamination of GHPs between the GH-secreting pituitary adenoma tissue and the normal pituitary tissue.

The above are merely preferred examples of the present disclosure, and are not intended to limit the present disclosure in any form. Any simple modifications, changes, and equivalent variations made to the above examples according to the technical essence of the present disclosure should fall within the protection scope of the technical solutions of the present disclosure.

What is claimed is:

1. A method for identifying a human growth hormone proteoform (hGHP) pattern biomarker, comprising:
S1. collecting an hGH-secreting pituitary adenoma tissue sample and a normal pituitary tissue sample, and lysing the tissues separately to extract two sets of tissue proteins;
S2. equally dividing each of the two sets of tissue proteins obtained in Si into two parts, and subjecting the two parts separately to two-dimensional gel electrophoresis (2DGE) to obtain a protein-containing 2D gel a and a protein-containing 2D gel b;
S3. subjecting the protein-containing 2D gel a obtained in S2 to western blotting to obtain a visualized polyvinylidene fluoride (PVDF) membrane;
S4. soaking the protein-containing 2D gel b obtained in S2 in a Coomassie brilliant blue (CBB) staining solution to obtain a CBB-stained 2D gel b; and soaking the protein-containing 2D gel a undergoing western blotting in S3 in a CBB staining solution to obtain a CBB-stained 2D gel a;
S5. scanning the visualized PVDF membrane obtained in S3 and the CBB-stained 2D gel b and the CBB-stained 2D gel a obtained in S4 to obtain digital images; importing the digital images into Bio-Rad PDQuest 2D gel image analysis software to quantify volumes of protein spots; and matching an immuno-positive western blotting spot with corresponding protein spots in the CBB-stained 2D gels a and b;
S6. subjecting proteins in 2D gel protein spots in the CBB-stained 2D gels a and b obtained in S4 that are corresponding to an immuno-positive western blotting spot in the visualized PVDF membrane obtained in S3 to protein digestion with trypsin; and subjecting a tryptic peptide mixture to extraction and then purification with a ZipTipC₁₈ microcolumn to obtain a purified tryptic peptide mixture;
S7. subjecting the purified tryptic peptide mixture obtained in S6 to matrix-assisted laser desorption ionization time-of-flight mass spectrometry (MALDI-TOF-MS) analysis, liquid chromatography/electrospray ionization tandem mass spectrometry (LC-ESI-MS/MS) analysis, or matrix-assisted laser desorption ionization double-time-of-flight tandem mass spectrometry (MALDI-TOF-TOF-MS/MS) analysis, to obtain peptide fingerprint (PMF) data and MS/MS data;
S8. inputting the PMF data and MS/MS data obtained in S7 into the Mascot search engine to search for proteins in the UniProt database for identification;
S9. calculating theoretical tryptic peptide masses of GH with a peptide mass tool, and aligning sequences of tryptic peptides of GH to theoretical sequences of GH precursor, mature GH, and GH splicing variants 1, 2, 3, and 4 to determine characteristic tryptic peptides of the GH precursor, the mature GH, and the GH splicing variants 1, 2, 3, and 4; and comparing the obtained characteristic tryptic peptides to each mass spectrum obtained in S7 to determine whether a GHP is derived from the GH precursor, the mature GH, or the GH splicing variants 1, 2, 3, or 4; wherein the amino acid sequence of GH precursor is set forth in SEQ ID NO: 1, the amino acid sequence of mature GH is set forth in SEQ ID NO: 2, the amino acid sequence of GH splice variant 1 is set forth in SEQ ID NO: 3, the amino acid sequence of GH splicing variant 2 is set forth in SEQ ID NO: 4, the amino acid sequence of GH splicing variant 3 is set forth in SEQ ID NO: 5, and the amino acid sequence of GH splicing variant 4 is set forth in SEQ ID NO: 6;
S10. subjecting the GH-secreting pituitary adenoma tissue and the normal pituitary tissue to quantitative phosphoproteomics: Briefly, subjecting proteins of the two tissues separately to protein digestion with trypsin, labeling a tryptic peptide mixture with an iTRAQ reagent, and enriching phosphopeptides with TiO₂; using LC-MS/MS analysis to identify an amino acid sequence and a phosphorylation site of a phosphoprotein and quantify an abundance of each phosphopeptide; and comparing an obtained tryptic peptide where a GH phosphorylation site is located to each mass spectrum obtained in S7 to determine a phosphorylation state of GHP;
S11. subjecting the GH-secreting pituitary adenoma tissue and the normal pituitary tissue to quantitative ubiquitinomics: Briefly, subjecting proteins of the two tissues separately to protein digestion with trypsin, and using ubiquitin antibodies to enrich ubiquitinated peptides from an obtained tryptic peptide mixture; using LC-MS/MS analysis to identify an amino acid sequence and an ubiquitination site of an ubiquitinated protein; using a label-free quantification method to quantify an abundance of an ubiquitinated peptide; and comparing an obtained tryptic peptide where a GH ubiquitination site is located to each mass spectrum obtained in S7 to determine an ubiquitination state of GHP; and
S12. subjecting the GH-secreting pituitary adenoma tissue and the normal pituitary tissue to quantitative acetylomics: Briefly, subjecting proteins of the two tissues separately to protein digestion with trypsin, and using acetyl antibodies to enrich acetylated peptides from an obtained tryptic peptide mixture; using LC-MS/MS analysis to identify an amino acid sequence and an acetylation site of an acetylated protein; using a label-free quantification method to quantify an abundance of an acetylated peptide; and comparing an obtained tryptic peptide where a GH acetylation site is located to each mass spectrum obtained in S7 to determine an acetylation state of GHP.


SEQUENCE LISTING (23 sequences)

SEQ ID NO: 1 - protein, 217 aa, Artificial Sequence
  Amino acied sequence of GH Precursor
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 2 - protein, 191 aa, Artificial Sequence [sequence omitted]

SEQ ID NO: 3 - protein, 191 aa, Artificial Sequence [sequence omitted]

SEQ ID NO: 4 - protein, 176 aa, Artificial Sequence
  Amino acid sequence of GH Splicing variant 2
FPTIPLSRLFDNAMLRAHRLHQLAFDTYQEFNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 5 - protein, 153 aa, Artificial Sequence
  Amino acid sequence of GH Splicing variant 3
FPTIPLSRLFDNAMLRAHRLHQLAFDTYQEFEEAYIPKEQKYSFLQNPQTSLCFSESIPTPSNREETQQKSNLELLRISLLLIQTLMGRLEDGSPRTGQIFKQTYSKFDTNSHNDDALLKNYGLLYCFRKDMDKVETFLRIVQCRSVEGSCGF

SEQ ID NO: 6 - protein, 145 aa, Artificial Sequence
  Amino acid sequence of GH Splicing variant 4
FPTIPLSRLFDNAMLRAHRLHQLAFDTYQEFEEAYIPKEQKYSFLQNPQTSLCFSESIPTPSNREETQQKSNLELLRISLLLIQSWLEPVQIFKQTYSKFDTNSHNDDALLKNYGLLYCFRKDMDKVETFLRIVQCRSVEGSCGF

SEQ ID NO: 7 - protein, 19 aa, Artificial Sequence
  Amino acid sequence of the tryptic peptide (46-64)
LHQLAFDTYQEFEEAYIPK

SEQ ID NO: 8 - protein, 21 aa, Artificial Sequence
  Amino acid sequence of tryptic peptide (121-141)
SVFANSLVYGASDSNVYDLLK

SEQ ID NO: 9 - protein, 8 aa, Artificial Sequence
  Amino acid sequence of the characteristic peptide (27-34)
FPTIPLSR

SEQ ID NO: 10 - protein, 26 aa, Artificial Sequence
  Amino acid sequence of the signal peptide (1-26)
MATGSRTSLLLAFGLLCLPWLQEGSA

SEQ ID NO: 11 - protein, 28 aa, Artificial Sequence
  Amino acid sequence of the characteristic peptide (7-34)
TSLLLAFGLLCLPWLQEGSAFPTIPLSR

SEQ ID NO: 12 - protein, 13 aa, Artificial Sequence
  Amino acid sequence of the tryptic peptide (172-184)
FDTNSHNDDALLK

SEQ ID NO: 13 - protein, 18 aa, Artificial Sequence
  Amino acid sequence of the tryptic peptide (167-184)
QTYSKFDTNSHNDDALLK

SEQ ID NO: 14 - protein, 22 aa, Artificial Sequence
  Amino acid sequence of the tryptic peptide (172-193)
FDTNSHNDDALLKNYGLLYCFR

SEQ ID NO: 15 - protein, 11 aa, Artificial Sequence
  Amino acid sequence of the acetylated peptide (161-171)
TGQIFKQTYSK

SEQ ID NO: 16 - protein, 5 aa, Artificial Sequence
  Amino acid sequence of the acetylated peptide (167-171)
QTYSK

SEQ ID NO: 17 - protein, 18 aa, Artificial Sequence
  Amino acid sequence of the acetylated peptide (154-171)
LEDGSPRTGQIFKQTYSK

SEQ ID NO: 18 - protein, 24 aa, Artificial Sequence
  Amino acid sequence of the acetylated peptide (161-184)
TGQIFKQTYSKFDTNSHNDDALLK

SEQ ID NO: 19 - protein, 27 aa, Artificial Sequence
  Amino acid sequence of the acetylated peptide (167-193)
QTYSKFDTNSHNDDALLKNYGLLYCFR

SEQ ID NO: 20 - protein, 29 aa, Artificial Sequence
  Amino acid sequence of the ubiquitnated peptide (68-96)
YSFLQNPQTSLCFSESIPTPSNREETQQK

SEQ ID NO: 21 - protein, 13 aa, Artificial Sequence
  Amino acid sequence of the ubiquitnated peptide (91-103)
EETQQKSNLELLR

SEQ ID NO: 22 - protein, 6 aa, Artificial Sequence
  Amino acid sequence of the ubiquitnated peptide (91-96)
EETQQK

SEQ ID NO: 23 - protein, 30 aa, Artificial Sequence
  Amino acid sequence of the ubiquitnated peptide (91-120)
EETQQKSNLELLRISLLLIQSWLEPVQFLR